(19) United States
(12) Patent Application Publication
(10) Pub. No.: US 2006/0015029 A1
(43) Pub. Date: Jan. 19, 2006

(54) Body image analysis system

(76) Inventor: Traci L. Metzger, Loveland, OH (US)

Correspondence Address: WOOD, HERRON & EVANS, LLP, 2700 CAREW TOWER, 441 VINE STREET, CINCINNATI, OH, 45202, US

(21) Appl. No.: 11/120,873

(22) Filed: May 3, 2005

Related U.S. Application Data
(60) Provisional application No. 60/567,875, filed on May 4, 2004.

(51) Int. Cl. A61B 5/05

(52) U.S. Cl. 600/407

(57) ABSTRACT

A method, apparatus and program product provide analysis and data resources by allowing a user to manipulate and track a scanned and/or displayed body image, providing an online self help interface that includes answers to anticipated questions, as well as a back-end statistical database useful in scientific analysis.

### CROSS-REFERENCE TO RELATED APPLICATION

This application claims benefit of priority to U.S. Provisional Application Ser. No. 60/567,875, filed on May 4, 2004 by Traci L. Metzger, which is incorporated herein in its entirety and is entitled “BODY IMAGE ANALYSIS SYSTEM.”

### FIELD OF THE INVENTION

The present invention relates generally to computer operations, and more particularly, to a computer implemented system for assessing and treating patient disorders.

### BACKGROUND OF THE INVENTION

Timely body image assessment and treatment can improve and even save the lives of those that suffer from low self esteem, eating disorders and other image related issues. While the response to such treatment has been tremendous, many persons in need of treatment go without it, in part due to a reluctance to seek out professional help in person. Such may be the case where a person is too self conscious, nervous, or busy to arrange for a visit.

Professionals trained in assessing and treating body image issues can be consequently handicapped in reaching people in need of their services. In addition to accessibility, these professionals conventionally face other obstacles, largely attributable to the cumbersome, overwhelming and growing volume of statistics, books and other studies that must be considered when manually assessing and/or treating a patient. Moreover, the clinical/scientific studies relied upon by professionals are often based on small pools of participants from a discrete geographic location(s), limiting the applicability of a study.

Consequently, and for in part the above delineated reasons, there exists a need for an improved manner of conducting body image analysis.

### SUMMARY OF THE INVENTION

The present invention provides an improved apparatus, method and program product for providing body image analysis. Such analysis may include assessment and treatment features that, for instance, allow a user to manipulate and a displayed body image. The body image may include a computer scanned or computer generated image of the user or another, and may be compared and tracked over time to reflect changes in a user's actual, perceived and/or desired appearance. Another feature of the invention may provide online answers and interactive tools configured to help a patient, health professional or other user in body image assessment. The display of such answers and tools may be prompted by the user answering a set of preliminary questions, the answer(s) to which the system may use to tailor the display. For instance, answers to anticipated questions may be automatically determined by the virtue of the system matching user input to an appropriate, stored set of answers.

In another respect, a feature of the analysis system may automatically and/or transparently compile statistical data based upon the interaction of the user. Such statistical data may have application in furthering research and treatment.

These and other objects and advantages of the present invention shall be made apparent from the accompanying drawing and the description thereof.

### DESCRIPTION

According to an image warping aspect of the invention, a user may manipulate the front, back and side view of either a silhouette image that is based on in individual's actual body measurements, or an uploaded digital image of an individual. Before the individual makes any changes to an image, the warping engine may automatically alter the image slightly in order to provide a “neutral” image for the individual to begin manipulating into the following states: (a) how the individual thinks s/he looks (perceived) (b) how s/he wants to look (ideal) and (c) what is a realistic way s/he should look (realistic). This image manipulation may occur by the user taking a mouse to the edge of a specific area of the body silhouette or digital image and dragging the section of the image to the shape s/he feels is what is perceived, ideal or realistic. The individual will have the opportunity to compare the images s/he has manipulated to see the discrepancy between their perceived, ideal, realistic and actual image. With permission from the individual, data will be captured on the discrepancies between the images for analysis and reporting.

Another embodiment consistent with the invention processes interactive user input to help prioritize the importance of different steps/phases of an assessment or treatment program. The steps/phases of the program may be prioritized based on how the multiple choice questions are answered. The customized program based on the individuals' answers will have the opportunity to address their specific needs first and then work through less critical sections after the priority sections are complete. Some sections will have to occur in a specific order to make the individual program successful, and are dependant upon the self-improvement program tailored to individual. With permission from the individual, data will be captured and analyzed for report generation.

According to another aspect of the invention, data is collected at a worldwide scale. Such data is generated from the use of Real-Time Body Warping Application and the Customized Self-Improvement Programs. Permission may be requested from individuals to collect data that is generated from the individual's session. Data will be stored in a secure database separate from identity-related information. A code will be assigned to the data as the only identifier to map data to the correct file if the individual returns multiple times to utilize their customized program or body-warping application. Identity-related information for billing reasons will be kept in a separate database. This will provide the opportunity to collect data worldwide to analyze and better understand trends of medical disorders of people that are not seeking medical assistance or are in need of additional support.

While the present invention has been described in terms of various embodiments and while these embodiments have been described in considerable detail, it is not the intention of the applicants to restrict or in any way limit the scope of the appended claims to such detail. For instance, additional disclosure is included in the appendix attached hereto, which is hereby incorporated by reference in its entirety. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicant's general inventive concept.

What is claimed is:

Definitions

The following definitions, acronyms, and abbreviations are found in tis document:

TABLE 1
Definitions

| Term | Definition |
|---|---|
| Internet | Worldwide network of computer networks that use the |
| | TCP/IP network protocols to facilitate data transmission |
| | and exchange |
| Web | A software program which allows the viewing of data |
| browser | transmitted by the Internet. Examples are Internet Explorer |
| | and Netscape. |
| Web- | A software application which is programmed to present data |
| based | by a Web browser that is transmitted by the Internet. The |
| applica- | application is hosted on a central server and not located on |
| tion | individual desktop computers. |
| Module | Individual programs within the application that operate |
| | separately, collect different data sets and have separate |
| | security measures. |
| Compo- | An element of a system/module |
| nent | |
| Web | An individual accessing the public sections of the Web site |
| visitor | that does not require user authentication through username |
| | and password. |
| Participant | An individual that has signed a release to use the application |
| | and allow the capture of specific data sets. |
| User | An individual that is facilitating the use of the application |
| | and its modules. |
| Profile | A file containing information of a patient/participant |
| Body | A discrepancy between a person's perceived image and their |
| Dis- | ideal image. |
| satisfac- | |
| tion | |
| Body | A discrepancy between a person's actual image and their |
| Distortion | perceived image. |

Introduction

Purpose

This document identifies both the business and technical requirements and specifies the functionality to be included in the prototype f. The current business problem and the objectives of the project, as well as functional and technical requirements are documented. This document, once it is approved, serves as the basis for:
Creating the detailed design
Monitoring progress during system development
Providing a successful implementation
Creating the acceptance test plan of the completed system

Scope

The scope of this project is to architect, design and build a prototype that will be used in pilot research studies to prove the validity of the PTI concept and the benefits it offers both the medical and non-medical community. Once the studies have proven the validity of the concept that is represented in the prototype, it will be tested for market response through public relations efforts.

Out of Scope

While important, the following items are beyond the scope of this initial effort.
Database development will only be to the extent that it can support the prototype. Once the prototype is proven valid in the marketplace, the complete front-end and back end application will be developed.

Assumptions
High priority requirements will be implemented in the prototype.
Medium priority requirements will be implemented in the prototype if time permits.
Low priority requirements will be implemented in the first full version release.

User Roles and Responsibilities

TABLE 2
User roles and responsibilities

| Market | User | Description |
|---|---|---|
| Clinical | Nurses, Medical Assistants, | Medical professionals |
| | Lab Technicians, | such as a physician, psychologist, |
| | Medical Interns | psychiatrist or therapist |
| | | will decide to have patient take |
| | | assessment(s). The users listed |
| | | to the left will administer the |
| | | assessment. |
| General | An individual with | Individual will pay a |
| Public | moderate Web skills | fee to access the online self-help |
| | | modules, enter measurements |
| | | to manipulate silhouettes |
| | | or to upload their actual |
| | | images to manipulate and |
| | | receive results. |

Requirement Sources

Requirement Gathering Methods

These requirements have been gathered through consultations with medical experts, extensive secondary research and applying that knowledge to develop the concept which is the basis for the prototype and launch of PTI.

Requirements

Functional Requirements

Prototype Functional Requirements

TABLE 3
Prototype Functional Requirements

| # | Requirement | Priority | Description |
|---|---|---|---|
| 1.01 | Choose Your Language - English or Japanese | High | Prototype will be test marketed in the |
| | | | U.S. and Japan. |
| 1.02 | I would like to: | High | Dropdown to access different modules |
| | | | of the application. Dropdown options |
| | | | may show all options or two to three |
| | | | based on audience of application. This |
| | | | could also be a nav bar. |
| 1.03 | Take a body image assessment | High | Up to five assessments that are 1-2 |
| | | | pages each. Multiple choice answers. |
| | | | This module will need to be available |
| | | | independently for university and |
| | | | clinical studies. Scores will need to be |
| | | | tabulated on separate page that user |
| | | | cannot see. Score and answers will be |
| | | | saved in a database which only tracks |
| | | | the UserID of the user. |
| 1.04 | Create a customized self-guided improvement program | High | Link to self-guide module |
| 1.05 | COMPAIR my silhouettes | High | Link to silhouette module |
| 1.06 | COMPAIR my actual images | High | Link to image module |
| 1.07 | Enter User ID and Password | High | User ID automatically generated and |
| | | | user sets up password. Information |
| | | | kept in a separate database with |
| | | | contact/billing information. This may |
| | | | take them to where the page they were |
| | | | working in a customized self- |
| | | | improvement module. Images will not |
| | | | be saved in COMPAIR so people will |
| | | | have to be warned that they will lose |
| | | | images if they close out but statistics |
| | | | will be saved on variances between |
| | | | different images and comparisons. |
| 1.08 | Friends and Family Information | Medium | Link to 2-3 pages of sample |
| | | | information that will be included to |
| | | | offer support, information and |
| | | | resources for friends or family |
| | | | members where a loved one has a |
| | | | medical illness or issues where body |
| | | | image is a factor. Can be incorporated |
| | | | into main nav bar. |
| 1.09 | Hidden URL | High | The URL must not be visible in a |
| | | | clinical setting to prevent patients from |
| | | | surfing the Web. |
| 1.10 | Assessment Overview | High | For prototype, content will describe |
| | | | the purpose of the assessment and the |
| | | | results that will be generated. In a |
| | | | medical facility, results will be |
| | | | generated in a separate password |
| | | | protected area of the site for a medical |
| | | | professional to view the results and |
| | | | make decisions in treatment. For the |
| | | | public, results will be generated with |
| | | | general descriptions of meaning of |
| | | | results after individual clicks on |
| | | | submit. |
| 1.11 | Dropdown of different assessments to select from | | Two-five assessments will be available |
| | | | for the prototype. During research |
| | | | studies, two will be pre-selected. |
| 1.12 | Assessment Results | | Results will likely be in ranges that will |
| | | | have descriptions associated with each |
| | | | range. This page needs to be secure in |
| | | | a medical setting so the medical |
| | | | professional can review results without |
| | | | the patient seeing the results |
| | | | themselves. General public will be |
| | | | able to see results. |
| 1.13 | Legal Disclaimer/Privacy Statement | | Legal description of what PTI will or |
| | | | will not do with information that is |
| | | | gathered. |
| 1.14 | Silhouette Overview - (This page is from the 1.05 link on the Home | High | Content to describe the purpose of the |
| | Page) | | silhouettes and what the user will do to |
| | | | create the silhouettes that reflects their |
| | | | current image. |
| 1.15 | Silhouette Demo | High | To provide a visual overview of what |
| | | | the user will experience by entering |
| | | | their measurements and manipulating |
| | | | the silhouettes. Flash will have the |
| | | | most impact obviously. Need to have |
| | | | another option for those that do not |
| | | | have flash in other countries. |
| 1.16 | Sign - up to create silhouettes | High | After seeing the demo, they will click |
| | | | on the register/sign up button to enter |
| | | | billing information and generate their |
| | | | automatic User ID and create their |
| | | | password. This info will be kept in |
| | | | Database #1. |
| 1.17 | Link to Registration Page | High | Links to page for public to sign up and |
| | | | enter billing and contact information. |
| 1.18 | Registration Form | Low | See fields in wireframe as a guide to |
| | | | types of fields that should be used. |
| | | | After username and password are |
| | | | issued and verified, the user will need |
| | | | to be directed to the module they |
| | | | purchased to start right away or |
| | | | directions to remind them how to log |
| | | | in. |
| 1.19 | UserID and Password Set up | Low | User registers information and then |
| | | | they are prompted with an |
| | | | automatically generated ID and enter |
| | | | their unique password. |
| 1.20 | Silhouette Measurements | High | Required measurements will be |
| | | | finalized once it is determined if a pre- |
| | | | existing software application will be |
| | | | used or if it will be a custom |
| | | | application. We would like the |
| | | | minimum number of required |
| | | | measurements to receive the most |
| | | | accurate silhouette image of the |
| | | | person. |
| 1.21 | Neutral Image | High | The actual image will appear “neutral” |
| | | | either slightly or significantly larger or |
| | | | smaller. The demo should indicate |
| | | | that the “neutral” image is random and |
| | | | will not always be slightly smaller for |
| | | | example. The computer program |
| | | | should randomly select how to |
| | | | manipulate the image. |
| 1.22 | Perceived Image - how the person “thinks” they look based on | High | The user will see “x”s or points on |
| | manipulation of a “neutral” image. | | sections of the neutral image and they |
| | | | can take their mouse and either drag |
| | | | the edge of the image to make it either |
| | | | narrower or wider to how they “think” |
| | | | or perceive they look. For example, |
| | | | they can modify the top area of their |
| | | | arm or the lower portion. Another |
| | | | example would be the waist being |
| | | | manipulated without affecting the hips. |
| 1.23 | Perceived Image Data Capture | High | The measurements of the Perceived |
| | | | image once complete will be compared |
| | | | against the Actual image that is based |
| | | | on real measurements. This data will |
| | | | be stored in Database #2. |
| 1.24 | Ideal Image | High | The images are randomly “neutralized” |
| | | | again and the user manipulates front, |
| | | | back and side images on how the want |
| | | | to look. |
| 1.25 | Ideal Image Data Capture | High | The measurements of the Ideal image |
| | | | will be compared against the Actual |
| | | | image that is based on real |
| | | | measurements. This data will be stored |
| | | | in Database #2. |
| 1.26 | Ideal vs. Perceived Image | High | Ideal vs. Perceived image demonstrates |
| | | | body image dissatisfaction. Data will be |
| | | | tracked to measure level of dissatisfaction |
| | | | in a standardized manner. |
| 1.27 | Front, Back and Side Buttons | High | Person can click on any three buttons |
| | | | to select the view the want to see. |
| 1.28 | Create customizable self-improvement program | High | Person will answer questions which |
| | | | will prioritize the modules that they |
| | | | will go through based on their |
| | | | individual needs. Data will be |
| | | | captured in database to analyze what |
| | | | people are seeking or feel they need to |
| | | | improve in their lives. Estimate is 5-10 |
| | | | questions. Questions will be multiple |
| | | | choice. |
| 1.29 | Patent-Pending | High | Patent-pending should be discrete, yet |
| | | | visible on every page. |
| 1.30 | Self-improvement Recommendation | High | After the person has answered the |
| | | | questions, the recommended modules |
| | | | will be shown in order of importance. |
| | | | Each module will have a brief |
| | | | description. These results should be |
| | | | captured in a database not associated |
| | | | with the user's personal information - |
| | | | just ID number. |
| 1.31 | Register for program | High | If person wants to move forward they |
| | | | will click on the register button to |
| | | | provide user and payment information |
| | | | if they did not select to do this along |
| | | | with their silhouette or image module. |
| 1.32 | Not at this time | High | After reviewing the program the |
| | | | person decides they do not want to |
| | | | purchase the program. This will need |
| | | | to be collected in a database. |
| 1.33 | Selecting To (on registration form) | High | The person has the option to purchase |
| | | | one module or multiple modules. They |
| | | | can complete one module and choose |
| | | | to go back and purchase another. They |
| | | | will keep the same UserID and |
| | | | Password as they had before. |
| 1.34 | Sample Module | High | For the prototype, show one page of a |
| | | | sample module. Does not need to |
| | | | track info in the database for the |
| | | | prototype. It does need to track all |
| | | | information provided in Version 1.0. |
| 1.35 | Friends and Family Information | High | For the prototype, one page of sample |
| | | | information and resources for friends |
| | | | and family. Version 1.0 will require this |
| | | | area to be database driven to easily |
| | | | update physician contact information |
| | | | for example. Will contain white papers, |
| | | | links to research, medical resources etc. |
| 1.36 | I would like to PREPAIR: | High | Modules for prevention of body image |
| | | | disturbances and eating disorders. |
| | | | Either a drop down or part of the nav |
| | | | bar with sub-level choices. |
| 1.37 | Parental Consent Form | Med | Not essential for the prototype but |
| | | | need to show it in the dropdown and it |
| | | | will be critical for Version 1.0. Will |
| | | | need to submit to Database #1 |
| 1.38 | PREPAIR: 9-11 years of age | Med | Show intro page in the prototype and |
| | | | complete modules developed with |
| | | | content and subject matter that is |
| | | | appropriate for 9-11 year old |
| | | | adolescents. |
| 1.39 | PREPAIR: 12-15 years of age | Med | Show intro page in the prototype and |
| | | | complete modules developed with |
| | | | content and subject matter that is |
| | | | appropriate for 12-15 year old |
| | | | adolescents. |
| 1.40 | PREPAIR: 16-18 years of age | Med | Show intro page in the prototype and |
| | | | complete modules developed with |
| | | | content and subject matter that is |
| | | | appropriate for 10-12th grade level. |
| 1.41 | End Assessment for both Silhouette, Digital Images and Customizable | High | User takes the same assessment they |
| | Self-guided Improvement Program | | did before they went through the |
| | | | silhouette or digital image module to |
| | | | see if they improved on their results. |
| | | | This is will guide the individual and |
| | | | provide data which will be captured in |
| | | | database #2. Some individuals my |
| | | | choose to take the beginning |
| | | | assessment and unless they decide to |
| | | | participate in the silhouette, digital |
| | | | image or customized self-guided |
| | | | improvement program, they will not |
| | | | be presented with the second |
| | | | assessment. (View 2.14 in Database |
| | | | specs) |
| 1.42 | Saving Digital Images | High | Images will only be saved under |
| | | | unique circumstances with an |
| | | | agreement with another |
| | | | facility/company requires that images |
| | | | are saved. A consent form for each |
| | | | person will need to be signed in order |
| | | | for this to occur and record that this |
| | | | consent form was complete must be |
| | | | filed with the user information in |
| | | | Database #1. Consider allowing user |
| | | | to save manipulated images to their |
| | | | desktop and then if they exit in the |
| | | | middle of the module, they can upload |
| | | | the images they manipulated so that |
| | | | effort is not lost and PTI does not save |
| | | | images. |
| 1.43 | Specs/Requirements for equipment to capture and upload digital | High | These specs will be determined upon |
| | images - need to consider using cell phones that take digital pictures | | either the pre-packaged software that is |
| | for countries like Japan. | | used or requirements of the custom |
| | | | application. |
| 1.44 | Consistency of Images | High | Evaluate the need for the angles of the |
| | | | arms and legs to be the same across all |
| | | | images to capture data that is |
| | | | consistent across different individuals. |
| | | | Prefer for it not to be. |
| 1.45 | Feedback/Questionnaire | Med | Please review the university study from |
| | | | Dr. Thomas Cash at Old Dominion |
| | | | University to identify the priority in the |
| | | | prototype. May want to consider |
| | | | getting feedback at the bottom of |
| | | | every page. Example of this is at the |
| | | | bottom of Microsoft help where they |
| | | | request feedback and it is automatically |
| | | | sent via Internet. Feedback is broken |
| | | | up throughout the site to avoid |
| | | | inconvenience of long form. |

Prototype Database #1 and #2 Requirements

TABLE 4
Prototype Database Requirements

| # | Requirement | Priority | Description |
|---|---|---|---|
| 2.01 | Database #1 will contain contact information and billing information of | High | Medical facilities will contact PTI to |
| | either an individual user or medical facility. | | set up an account or work with a PTI |
| | | | sales representative. Individuals will |
| | | | enter their information via the online |
| | | | application. |
| 2.02 | Database #1 will have user ID number associated individual's file or | High | Someone may register and then want |
| | group of ID numbers associated to a medical facility. | | to come back at another time to start |
| | | | the module. When they log in with |
| | | | their username and password, the |
| | | | system will need to identify what they |
| | | | purchased so they do not have access |
| | | | to other modules and just the one they |
| | | | purchased. |
| 2.03 | Database #1 will store password generated by the individual or medical | High | |
| | facility. | | |
| 2.04 | Web application will have a “Forgot My Username/Password” function | Medium | Medical facilities will require some |
| | which will email this information to the individual or medical facility. | | additional level of authentication when |
| | | | requesting this information to prevent |
| | | | patients or other individuals accessing |
| | | | this information. |
| 2.05 | Database #1 will record use of application and track modules used for | Low | |
| | billing purposes for medical facilities. | | |
| 2.06 | Database #1 will provide activity reports containing data such as: activity | Low | |
| | per module, estimated time of use for each session etc. | | |
| 2.07 | Databases for prototype should be able to store 150-200 records. | High | |
| 2.08 | Databases for V 1.0 should store 125,000 records for Year 1; 175,000 | Low | |
| | records for Year 2; 225,000 records for Year 3. | | |
| 2.09 | Database #2 will not contain individual or patient information. It will | High | Only build for the 150 to 200 records |
| | contain the UserID and Password which will be the map to the other | | that will be developed for the |
| | database for billing information. | | prototype. |
| 2.10 | Database #2 will store measurement information based on UserID and | High | For example, measurements to |
| | session number. | | develop the silhouette images and the |
| | | | difference between Actual and Ideal, |
| | | | Actual and Perceived, Actual and |
| | | | Realistic and Perceived and Ideal. |
| | | | Metric measurements will need to have |
| | | | the option to convert to English |
| | | | measurement in inches for example. |
| 2.11 | Parental Consent Form | High | Track parents that have given or not |
| | | | given consent for them to have their |
| | | | children use the PREPAIR or |
| | | | COMPAIR modules. This will be |
| | | | important to show in the prototype. |
| 2.12 | Exiting the module mid-stream | Low | If someone ends midstream, images |
| | | | will not be stored but the statistics will. |
| | | | If the user can pick up where they left |
| | | | off that will be beneficial. Not a |
| | | | priority for the prototype but will be |
| | | | for Version 1.0. |
| 2.13 | Database #2 - Measurement/Findings Reports | Med | Will need to develop sample report(s) |
| | | | for the prototype but not the complete |
| | | | reporting system. Reports will need to |
| | | | be convertible between English and |
| | | | Metric measurements. In Japanese and |
| | | | English. |
| 2.14 | Beginning and end assessment data captured per UserID Number | Med | Please review proposed university |
| | | | study to identify the level of |
| | | | importance in the prototype phase. |
| | | | User takes the same assessment they |
| | | | did before they went through the |
| | | | silhouette or digital image module to |
| | | | see if they improved on their results. |
| | | | This is will guide the individual and |
| | | | provide data which will be captured in |
| | | | database #2. Some individuals my |
| | | | choose to take the beginning |
| | | | assessment and unless they decide to |
| | | | participate in the silhouette, digital |
| | | | image or customized self-guided |
| | | | improvement program, they will not |
| | | | be presented with the second |
| | | | assessment. |
| 2.15 | Consent form to save images | High | Database #1 - important to keep |
| | | | record of these consent forms and |
| | | | images will only be saved in a detailed |
| | | | agreement with a medical facilitate or |
| | | | business that required this function. |

PTI Prototype Application System & Security Requirements

TABLE 5
Risk Consulting Home Page Requirements

| # | Requirement | Priority | Description |
|---|---|---|---|
| 3.01 | No access to a Web browser in a medical environment | High | If possible, the application should not |
| | | | require opening a browser, typing in an |
| | | | address to access the Home page. If a |
| | | | browser is available on the Desktop, it |
| | | | allows the opportunity patients will |
| | | | access the browser and surf the Web. |
| 3.02 | Desktop Icon to Application | High | Medical facility will click on a desktop |
| | | | icon which will link them directly to |
| | | | the Web-based application. They will |
| | | | log into the application prior to use |
| | | | and enter in the patients UserID and |
| | | | Password. When patient enters the |
| | | | application will be ready for use. |
| 3.03 | Credit Card and Purchase Order Payment Abilities | Low | System will need to be able to accept |
| | | | major credit cards and take purchase |
| | | | order information from medical |
| | | | facilities. |
| 3.04 | Database and Application Security | High | Implementation of advanced database |
| | | | and application security is critical to |
| | | | protect data. |
| 3.05 | Measurement variances by country - metrics vs. inches/feet etc. | High | Japan will enter different |
| | | | measurements and we will need to |
| | | | convert the data to the same |
| | | | measurement standard to have |
| | | | consistency in data that is collected. |
| 3.06 | Measurement ranges | High | PTI will establish what measurements |
| | | | ranges are not within general ranges to |
| | | | avoid capturing grossly incorrect |
| | | | measurements entered by users. |

Other Functional Requirements

TABLE 4
Other Functional Requirements

| # | Requirement | Priority | Description |
|---|---|---|---|
| 6.01 | None at this time | n/a | n/a |

Technical/Infrastructure Requirements

TABLE 5
Technical/Infrastructure Requirements

| # | Requirement | Priority | Description |
|---|---|---|---|
| 6.01 | All authenticated users can access modules | High | |
| | via the Internet | | |
| 6.02 | Acceptable performance via dialup | High | |
| | connection | | |
| 6.03 | Internet Explorer 6.0 and above is | High | |
| | standard browser | | |

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

### Appendix B

General information will be entered into the system such as sex, race, age, height, weight, current diet, exercise habits, health status, and emotional well-being to open a new file. This will provide identification when updating files of specific clients, as well as create the ability to gather statistical data for market and medical research.

The software will begin with an option of three body-image questionnaires depending whether the client is a woman, man or adolescent. With permission, established questionnaires will be used in the software.

The software will be based on four images:
1) Actual
2) Perceived
3) Ideal
4) Realistic

The Actual, Perceived and Ideal Images will be utilized to determine a person's body dissatisfaction and distortion. These two terms are defined below:

Body Dissatisfaction: a discrepancy between a person's perceived image and their ideal image.

Body Distortion: a discrepancy between a person's actual image and their perceived image.

The person will have their front, side and back image scanned in to the computer using a digital camera. Before viewing their actual image, they will manipulate their Perceived and Ideal Images. For the person to determine their body distortion and dissatisfaction, they need a “neutral” image of themselves to manipulate. Their neutral image is created by minor automatic changes on certain areas of the body by the computer. For example, their thighs will be slightly smaller and their arms slightly larger. These automatic changes will be minimal so the person is not shocked when they see this “neutral” image. The neutral image will initially be blurred on the screen. It will be brought into focus with the minor changes. The blurred image will create the effect of the image being altered as it comes into focus.

The Perceived and Ideal Images will be manipulated individually with the front, side and back image appearing separately. The therapist/counselor and client will discuss their thoughts associated with each image by clicking the mouse and the image will alter in size a small increment. The image will be manipulated altering the circumference of specific body parts. For example, the upper part of the leg from the hip down to the knee would be one section. Then another section would be from the knee down to the foot. Often a participant has issues about certain body parts. This will allow the participant to discuss these areas separately.

When the Perceived and Ideal images have been created, they will be compared individually to the client's Actual Image. During comparison, there will be six images on the screen. The front, back and side view will be displayed for the Perceived or Ideal Image and their Actual Image. As a comprehensive view, the Perceived, Ideal and Actual Images of either the front, back or side view can be displayed showing the differences between the three images.

The measurements of distortion will not be in “inches” because the patient may focus about reaching a certain measurement instead of what is realistic for that individual. Statistics are not needed for these images because it is visual. The distance between a person's Actual Image and maximum point of distortion from the revised image will be preconfigured; for example, 1 through 5. There will be a separate table for level of distortion and dissatisfaction.

Showing the tables may disturb some people to see their level of distortion and dissatisfaction. The numbers and table will not appear with the images so the patient will not see the number and the corresponding explanation.

The therapist/counselor will have the option of creating a Realistic Goal Image with the patient. They can discuss issues while the patient and therapist/counselor create the Realistic Goal Image together. By clicking the mouse, they can manipulate various body parts for an overall Realistic Goal. It will be important to emphasize this Realistic Goal Image is not based on an individual's statistics. Its purpose will be for the therapist/counselor and client to have a “tool” to discuss and determine what is realistic and not what will actually occur over time.

For motivational purposes, the Realistic Goal Image will be created for a result that the client can obtain in a short period of time. By providing an intermediate goal instead of the final Realistic Image initially, it will greatly increase the chances of the client being successful creating positive motivation. For example, if a client needs to convert to a healthy lifestyle and a factor in that is losing inches, an intermediate goal will be more attainable that giving the client the total inches.

These images can be saved for tracking progress throughout treatment. A file will be created for each client if the client signs a release. The therapist/counselor will be able to make notes on each session along with the images.

By using a projector, an option is for the therapist/counselor can project the client's image on to the wall. This will create an image for the patient that is “life-like” making the images even more realistic. This can be a tremendous advantage for the person to be looking at themselves instead of a blank silhouette without their features and personality.

A database will be created to gather data captured from of this module. Companies, universities and medical centers will download profiles of clients and their statistics to the database. Most studies are based at a university or medical center and have a limited participant population. This will provide the opportunity to gather data on a large cross-section of the population through leveraging the Internet and the World Wide Web.

Optional Component(s): Dietary and exercise modules can be developed and customized for weight loss centers and fitness centers by PTI to serve the needs of individual clients.

Listed below are examples of data that can be extrapolated from the profiles and the benefits it offers. These examples are not scientific-based or supported by a specific university or medical research facility.

Determination: Pinpoint when peer pressure, media and society begin to affect an adolescent and their self-esteem and self-perception.

Data: Utilize the gender, age range and adolescent body-image questionnaire

Benefits: School programs can be developed to address these issues with adolescents at the correct age before they typically develop.

Determination: Whether African-American women/men accept their bodies more than Caucasian women/men.

Data: Utilize race, gender and body-image questionnaire results.

Benefits: Gain a further understanding on different cultures and the effects it has on self-perception.

Determination: Whether women become more comfortable with their bodies as they get older and how media and society affects their self-perception.

Data: Utilize age, gender and answers from certain questions in the body-image questionnaire.

Benefit: Further understand reasons why women's perceptions change at a certain age and focus on developing programs to support women in age ranges that are still highly impacted by media and society.

Determination: An individual's self-perception and the stage of an eating disorder.

Data: Utilize body-image questionnaire, gender, age, body images and notes from specific time frames of treatment.

Benefit: Gain a deeper understanding how body-image and eating disorders relate with one another. With a better understanding medical facilities will be able to more effectively treat these disorders.

[image]

[image]

[image]

[image]

[image]

[image]

[image]

1. A computer implemented method for analyzing a body image of a user, comprising:
creating an electronic image of a body;
adjusting the electronic image of the body according to user input; and
storing the adjusted electronic image for comparison to another image.

2. The computer implemented method of claim 1, further comprising storing results related to the comparison for use in statistical research.

3. A computer implemented method for analyzing a body image of a user, comprising:
prompting the user for an answer to a predetermined question;
automatically determining a phase of an improvement program based upon the answer; and
outputting the phase for use in analyzing the body image of the user.

4. The computer implemented method of claim 3, further comprising storing the answer within a database for use in statistical research.
